SEQUENCE LISTING (4 sequences)

SEQ ID NO: 1 - protein, 115 aa, Homo sapiens
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 2 - protein, 4 aa, Artificial sequence
  CCK-4 peptide
WMDF

SEQ ID NO: 3 - protein, 8 aa, Artificial sequence [sequence omitted]

SEQ ID NO: 4 - protein, 97 aa, Homo sapiens
MVFVRRPWPALTTVLLALLVCLGALVDAYPIKPEAPREDASPEELNRYYASLRHYLNLVTRQRYGKRDGPDTLLSKTFFPDGEDRPVRSRSEGPDLW

(19) United States
(12) Patent Application Publication
(10) Pub. No.: US 2007/0265598 A1
(43) Pub. Date: Nov. 15, 2007

(54) Device and Method for Treating Weight Disorders

(75) Inventor: Yael Karasik, Ramat-HaSharon (IL)

Correspondence Address: Martin D Moynihan;Prtsi Inc, P O BOX 16446, Arlington, VA, 22215, US

(73) Assignee: Duocure, Inc., Ramat-Ha Sharon (IL)

(21) Appl. No.: 11/663,222

(22) PCT Filed: Sep. 29, 2005
(86) PCT No.: PCT/IL05/01053

§ 371 Date: Mar. 20, 2007

Related U.S. Application Data
(60) Provisional application No. 60/614,421, filed on Sep. 30, 2004.

(51) Int. Cl. A61K 9/22

(52) U.S. Cl. 604/891.1

(57) ABSTRACT

An apparatus and a method for treating a weight disorder in a subject are provided. The apparatus comprising an implantable device such as an inflatable balloon and electrodes capable of sensing a physiological change associated with food ingestion or hunger and a mechanism adapted for directly stimulating a region such as the duodenum which is responsive to a gastrointestinal satiety agent, such a mechanism can be a drug reservoir containing a drug such as CCK or analogs thereof which is contained within an inflatable balloon being implantable in a stomach of the subject. The apparatus and method provided here combine synergistic approaches to limiting meal size, i.e., chemo and mechano receptor activation of vagal satiety stimuli, electric stimulation of specific vagal pathways and limitations of gastric space.

### FIELD AND BACKGROUND OF THE INVENTION

The present invention relates to the treatment of obesity and, more particularly, to an apparatus comprising a device capable of sensing food ingestion or hunger and a mechanism for curbing appetite and/or limiting meal size. The invention exploits the physiologic understanding of appetite control by the gastrointestinal (GI) hormones and provides a method and apparatus which mimic the natural GI appetite control elements in a timely fashion at their exact locus of action.

Obesity is a prevalent disease that causes or exacerbates a large number of health problems. Obesity is a state of increase fat mass leading to deleterious metabolic effects. One of the more accepted parameters worldwide for obesity is the body mass index (BMI, kg/m²). The global epidemic of obesity, observed both in the developed and developing world is a major public health threat. In North America and most European countries rates of obesity have doubled within a generation, with half the adult population being overweight if not obese. In the Caucasian population, obesity is referred to a BMI above 30 kg/m², severe obesity as BMI>35 kg/m² and morbid obesity as BMI>40 kg/m².

Obesity causes or exacerbates a large number of health problems, both independently and in association with other diseases. In particular, it is associated with the development of type 2 diabetes mellitus, hypertension, pulmonary hypertension, coronary heart disease, stroke, an increased incidence of certain forms of cancer such as breast cancer and colon cancer, depression, obstructive sleep apnea, somnolence, and osteoarthritis of large and small joints. Consequently, the resource impact of obesity on global health care systems is enormous.

Though obesity can result from specific genetic defects it represents in most cases failure of the natural weight control elements amidst easy access to high caloric dense food and the cultural and personal life style of the current Western life style.

Food intake is governed by the drive to acquire energy (i.e., hunger) and the satisfaction of this drive (i.e., satiation). Once food is consumed, mechano-receptors and chemoreceptors in the gut detect the distension of the gut lining and the composition of food consumed. As a result, satiety factors such as cholecystokinin (CCK), bombesin, gastrin releasing peptide (GRP), glucagon, glucagon like peptide (GLP-1) and enterostatin are released control the food digestion and inform the CNS that the stomach is full and the gut contains nutrients (de Graaf C, et al., 2004, Am. J. Clinical Nutrition 79: 946-961). Of interest especially regarding hunger are signals like the hormone Ghrelin which are secreted by the stomach in the absence of and suppressed by food ingestion.

Endogenous CCK is a gastrointestinal peptide (with a length that varies between from 4 to 58 amino acids) found in both secretory and neural tissues. CCK is produced within the duodenal and jejunal mucosa and is secreted upon stimulation with intestinal nutrients. Two types of CCK receptors have been identified: CCK-α, a peripheral or alimentary sulfate-dependent binding subtype and CCK-β, for central or brain non-specific binding. In most species, basal plasma concentrations of CCK are around 1 pM and rise to 5-8 pM following food ingestion. However, it has recently been understood that locally released CCK in the duodenum and jejunum activates vagal afferent fibers in the absence of significant CCK plasma elevations and be dominantly responsible for CCK major effects i.e., pancreatic secretion and activation of the spincter of Oddi to release bile.

CCK is a major meal terminator. Exogenous CCK was found to adjust eating behavior in a manner consistent with a satiety enhancing action in animals and humans, while antagonizing the action of endogenous CCK reverses both the hypophagic and satiety enhancing effects of CCK (Moran T H and Kinzig K P, 2004, Am. J. Physiol. Gastrointest Liver Physiol. 286: G183-8). CCK-8 (Octapeptide CCK) retains the full activity of CCK and exerts similar effects on food intake and satiety in humans. Similarly, the CCK-antagonist, loxiglumide, was shown to reverse the inhibitory effect of both exogenous CCK-8, and fat induced endogenous CCK release, on the subjective feeling of appetite such as hunger and fullness. A direct effect of CCK-α agonism was shown to reduce pre-meal appetite and meal intake in lean humans. In addition, high doses of exogenous CCK administration were found to inhibit gastric emptying while CCK-α antagonists accelerate gastric emptying. (Ramkumar D., et al., 2003, Current Opinion in Gastroenterology, 19: 540-545). Thus, a major satiation effect of CCK probably results from its action on gastric emptying. CCK is released upon food entrance into the duodenum. This leads to increased neural activity in the gastric vagal afferents, stomach relaxation, constriction of the pylorus and inhibition of gastric emptying and increased gastric distension.

Interestingly, CCK directly activates vagal afferent fibers near or at the site where it is secreted. CCK-induced activation was demonstrated in both gastric and duodenal mechano-receptive fibers where CCK mimics and adds the action of distention and sensitizes fibers to subsequent distension. CCK interacts and activates the CCK-α receptors localized at the circular muscle in the pyloric sphincter resulting in pyloric contraction, inhibition of transpyloric flow, and slowing of gastric emptying.

Of interest are several observations showing the combined additive effects of gastric distention and satiety factors (Harry R., et al., 2003, Am. J. Physiol. Regul. Integr. Comp. Physiol. 285: R992). In both monkeys and humans effects of exogenous CCK were more pronounced after water or liquid meal preload. In humans CCK had a much larger effect on liquid diet consumption when administered to patients whose stomach was distended with a water filled balloon

The management of overweight and obesity is directed primarily to reduce energy intake and increase energy expenditure. There are numerous strategies that can be used to induce negative energy balance and short term weight loss. However, due to the chronic and relapsing nature of obesity, it is the long term efficacy of these approaches on maintaining lowered weight (and minimizing the risk of related chronic diseases) that is of fundamental importance

The first approach of treating obesity is lifestyle modification. This is based on the cognitive will of the obese individual. Though this is the most physiologic way it suffers from a high long term failure. Life style modification consists of restricted dietary treatment, increased physical activity program and behavior management.

Another approach is based on the oral administration of anti obesity drugs. These include drugs that act on the gastrointestinal system and interfere with nutrient metabolism such as lipase inhibitors (Pilichiewicz, A. et al., 2004, Am J Physiol Regulatory Integrative Comp Physiol., 287: R524-R533) and those that act on both the GI tract and the central nervous system to primarily suppress appetite (e.g., CCK or CCK analogs, GLP-1 or PYY). However, since these are peptides which are ruined by the gastric acidity and pepsinogens, their oral delivery is limited, resulting in low and inefficient levels in the intestine. Moreover, due to the unpredictable course of drug absorption in oral delivery, it is impossible to use these agents in a timed fashion with meals. Other drugs such as beta 3 adrenergic agonists [e.g., CL 316243; White C L., et al., 2004, Physiol. Behav. 82(2-3): 489-96], antagonists to the cannabinoid receptor (Lichtman A H and Cravatt B F, 2005, J. Clin. Invest. 115: 1130-3) and fat derived weight maintaining drugs (e.g., leptin) promote energy expenditure through both appetite control and the inducement of thermogenesis. Altogether, such drugs leads have shown minimal efficacy in the ability to reduce weight in significant percents, over a long and sustainable period of time, especially in the morbidly obese. Moreover, all of the drugs have various, and sometimes serious, side effects depending on their mechanism of action. For example, diarrhea for agent that limit food ingestion (e.g., orlistat) and high blood pressure and insomnia in drugs that activate the adrenergic system (sibutramine).

For obese patients at high risk of weight-related illness, and for the morbidly obese, there are a variety of available bariatric treatments. The most aggressive procedures are the various bariatric surgeries for reducing or bypassing the stomach or additional parts of the GI tract lumen. These surgeries include gastroplasty, gastric banding, intragastric balloons and gastric stapling. These methods can be highly effective because they severely limit the amount of food a person can ingest at one sitting, and depending upon the procedure, may rapidly induce a continual sense of satiety. However, such surgeries are associated with numerous immediate and late complications, which may lead to morbidity as well as mortality.

In another approach utilizing minor surgery, an intragastric balloon can be positioned by way of permanently placed, percutaneous endoscopic gastrostomy tube. However, as with any permanent aperture made through the skin, special hygienic practices are required of the patient and complications often arise. In addition, in the long-term, these procedures often fail since patients consume high caloric liquids, to overcome the mechanical restrictions posed by the procedure. Another approach involves an endoscopic procedure in which an intragastric inflatable balloon is placed via the esophagus. This procedure produces a feeling of fullness which limits food consumption to some degree. Another surgical procedure entails wiring a patient's jaw shut to limit food intake. However, besides being embarrassing and highly uncomfortable, this procedure carries an attendant risk of aspiration of vomit so patients must carry scissors or wire cutters at all times. However, although the current device and surgical approaches generally limit the patient ability to consume food, they do not address the drive to eat, thus resulting in poor compliance, success rates and overall frustration. Many patients outsmart the restriction by over-consuming high energy liquid diets, resulting in failure of weight loss.

Outside of the discipline of bariatrics and related procedures, many patients with chronic illnesses or illnesses which must be treated over a prolonged period of time (months or weeks), must remember to administer oral medicaments on a frequent and periodic schedule. Although the consequences of the disease may be serious and even life threatening, many patients find it difficult to rigorously comply with their prescribed long-term dosing regimen. A number of alternative drug delivery technologies have been developed to address this issue. Time-released oral medicaments are one alternative which can lessen the number of pills a patient must ingest daily. However, time-released medicaments normally cannot deliver a drug beyond the duration of a single digestive cycle, and in some cases are ineffective because of inactivation that occurs in stomach. Transdermal patches are another alternative. However, only a subset of drugs are compatible with this method and some patients suffer adverse reactions to the adhesives employed with same. Still further, while portable automated syringes can deliver intravenous drugs over long durations, this method however, is viewed as significantly lessening the quality of life and has inherent risks. Consequently, it is typically indicated only for treating very serious illnesses.

Altogether, the currently practiced therapies such as lifestyle modification, drug therapy and surgery accomplish little long-term success and call for an innovative physiologic approach to limit food intake and curb appetite.

Various patent applications have suggested sensing food intake by measuring physiological parameters that change as a function of food intake and reducing appetite by electrical stimulation (see for example, US Pat. Appl. Nos. 20050096637A1 and 20040059393).

To date, there is no method or medical device which successfully uses the understanding of the physiology of appetite control and aims to use them in order to address both the drive to eat and the ability to consume in the right temporal relations.

There is thus a widely recognized need for, and it would be highly advantageous to have, a method and a device for preventing and/or treating obesity devoid of the above limitations.

### SUMMARY OF THE INVENTION

According to one aspect of the present invention there is provided an apparatus for treating a weight disorder in a subject comprising an implantable device capable of sensing a physiological change associated with food ingestion or hunger and a mechanism adapted for directly stimulating a region responsive to a gastrointestinal satiety agent.

According to another aspect of the present invention there is provided a method of treating a weight disorder comprising: (a) implanting in a subject in need thereof a device capable of sensing a physiological change associated with food ingestion or hunger; and (b) functionally associating with the device, a mechanism adapted for directly stimulating a region responsive to a gastrointestinal satiety agent.

According to yet another aspect of the present invention there is provided an apparatus for treating a weight disorder in a subject comprising an implantable device capable of sensing a physiological change associated with food ingestion or hunger and a mechanism adapted for directly stimulating a region responsive to a gastrointestinal satiety agent, the mechanism comprises an inflatable balloon being implantable in a stomach of the subject and a drug reservoir containing a drug capable of stimulating the region, the drug is selected from the group consisting of CCK, a CCK analog, a CCK receptor agonist, a PYY, a PYY analog, GLP-1, a GLP-1 analog and oxyntomudulin.

According to still another aspect of the present invention there is provided a method of treating a weight disorder comprising: (a) implanting in a subject in need thereof a device capable of sensing a physiological change associated with food ingestion or hunger; and (b) functionally associating with the device a mechanism adapted for directly stimulating a region responsive to a gastrointestinal satiety agent, the mechanism comprises an inflatable balloon being implantable in a stomach of the subject and a drug reservoir containing a drug capable of stimulation of the region, the drug is selected from the group consisting of CCK, a CCK analog, a CCK receptor agonist, a PYY, a PYY analog, GLP-1 and GLP-1 analog.

According to an additional aspect of the present invention there is provided an apparatus for treating a weight disorder in a subject comprising a distending object being implantable in a stomach of the subject and a drug reservoir containing a drug capable of stimulation a region responsive to a gastrointestinal satiety agent.

According to further features in preferred embodiments of the invention described below, stimulation of the region is effected using a drug.

According to still further features in the described preferred embodiments the mechanism comprises a drug reservoir being capable of containing and releasing the drug in response to the physiological change associated with food ingestion or hunger.

According to still further features in the described preferred embodiments the apparatus further comprising an inflatable balloon implantable within a stomach of the subject, the inflatable balloon capable of activating vagal mechanoreceptors and/or space-filling.

According to still further features in the described preferred embodiments the method further comprising implanting in the subject an inflatable balloon capable of activating vagal mechanoreceptors and/or space-filling.

According to still further features in the described preferred embodiments the device comprises an inflatable balloon.

According to still further features in the described preferred embodiments the device comprises at least one electrode.

According to still further features in the described preferred embodiments the at least one electrode is disposed on a balloon implantable within a stomach of the subject.

According to still further features in the described preferred embodiments the mechanism further comprising at least one electrode capable of vagal innervation

According to still further features in the described preferred embodiments the at least one electrode capable of vagal innervation is disposed on a balloon implantable within a stomach of the subject.

According to still further features in the described preferred embodiments the mechanism further comprising a pump for releasing the drug from the drug reservoir.

According to still further features in the described preferred embodiments the pump is an osmotic pump, a mechanical pump and/or an electrical pump.

According to still further features in the described preferred embodiments the drug is released to a duodenum wall.

According to still further features in the described preferred embodiments the drug is released to an antral sphincter and/or an gastrointestinal wall.

According to still further features in the described preferred embodiments the at least one electrode is capable of sensing an electrical activity of a muscle.

According to still further features in the described preferred embodiments the implantable device capable of sensing the physiological change associated with food ingestion or hunger comprises a muscle activity sensor.

According to still further features in the described preferred embodiments the drug is a satiety drug and/or an anti food absorption drug.

According to still further features in the described preferred embodiments the satiety drug is selected from the group consisting of a CCK, a CCK analog, a CCK receptor agonist, a PYY, a PYY analog, GLP-1, GLP-1 analog and oxyntomudulin.

According to still further features in the described preferred embodiments the anti food absorption drug is a lipase inhibitor.

According to still further features in the described preferred embodiments the drug reservoir being positioned inside an inflatable balloon.

According to still further features in the described preferred embodiments the drug reservoir being implanted subcutaneously.

According to still further features in the described preferred embodiments the drug reservoir being implanted in a stomach of the subject.

According to still further features in the described preferred embodiments the drug reservoir being implanted subcutaneously.

According to still further features in the described preferred embodiments the drug reservoir being attached on the skin.

According to still further features in the described preferred embodiments the drug is coordinated with the device capable of sensing the physiological change associated with food ingestion or hunger, such that the drug is released following the sensing of the food ingestion or hunger.

According to still further features in the described preferred embodiments the release of the drug is effected by a bolus injection of the drug.

According to still further features in the described preferred embodiments the bolus injection is effected following 1-5 minutes of the sensing.

According to still further features in the described preferred embodiments the release of the drug is effected for a predetermined time period selected from the range of 1-60 minutes following the bolus injection.

According to still further features in the described preferred embodiments the vagal innervation commences 1-5 minutes following the sensing.

According to still further features in the described preferred embodiments the vagal innervation is effected for a predetermined time period selected from the range of 1-60 minutes.

According to still further features in the described preferred embodiments the device being implanted in a stomach of the subject.

According to still further features in the described preferred embodiments the mechanism comprises an injectable device capable of injecting a drug.

According to still further features in the described preferred embodiments the weight disorder is selected from the group consisting of obesity, bulimia, diabetes-related obesity, metabolic syndrome.

According to still further features in the described preferred embodiments a release of the drug is effected by a dispersing tube.

According to still further features in the described preferred embodiments the dispersing tube is selected capable of contacting a mucosal wall.

According to still further features in the described preferred embodiments the drug reservoir is positionable in the inflatable balloon.

According to still further features in the described preferred embodiments the distending object comprises an inflatable balloon.

The present invention successfully addresses the shortcomings of the presently known configurations by providing a method and an apparatus for treating weight disorders.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates one embodiment of the apparatus according to the present invention.

FIG. 2 illustrates another embodiment of the apparatus according to the present invention.

FIGS. 3a-b the duodenal dispersing tube according to one embodiment of the apparatus according to the present invention.

### DESCRIPTION OF THE PREFERRED EMBODIMENTS

The present invention is of an apparatus comprising an implantable device capable of sensing a physiological change associated with food ingestion or hunger and a mechanism adapted for directly stimulating a region responsive to a gastrointestinal satiety agent. Specifically, the present invention teaches a combination of appetite curbing agents delivered in a timely fashion to their natural location, activation of mechano receptors and/or bariatric therapy which can be used to treat weight disorders such as obesity and bulimia by mimicking the physiological control over satiety and coordinating the local release of satiety drugs such as CCK or analogs thereof to the duodenum immediately following sensing of food ingestion or hunger.

The principles and operation of the apparatus according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Obesity is a prevalent disease world-wild that causes or exacerbates a large number of health problems both independently and in association with other diseases. In particular, it is associated with the development of type 2 diabetes mellitus, hypertension, pulmonary hypertension, lipid disorders, coronary heart disease, stroke, an increased incidence of certain forms of cancer such as breast cancer and colon cancer, depression, obstructive sleep apnea, somnolence, and osteoarthritis of large and small joints.

The management of overweight and obesity is directed primarily to reducing energy intake and increasing energy expenditure. Numerous strategies are currently used to induce negative energy balance and short term weight loss. These include life style modification, oral administration of anti obesity or anti food absorption drugs, bariatric surgeries such as gastroplasty, gastric banding and gastric stapling, and implantation of intragastric balloons. However, while oral administration of anti-obesity drugs is inefficient, the currently used devices and surgical approaches generally limit the patient ability to consume food, but do not address the drive to eat, thus resulting in poor success rates and an overall frustration.

U.S. Pat. Appl. No. 20050085923 to Levine et al., discloses an anti obesity device for limiting food absorption.

U.S. Pat. Appl. No. 20030096785 discloses agents and methods for modulating the expression and activity of two G-protein coupled receptors (GPR12 and GPR3) which are involved in regulation of food intake in mammals.

U.S. Pat. Appl. No. 20050096637A1 discloses a device capable of measuring physiological parameters that change as a function of food intake, such as a core body temperature, and a drug delivery device for insulin or other substance capable of regulating the level of glucose in the patient.

U.S. Pat. Appl. No. 20040059393 discloses a method and an apparatus for regulating eating habits using a sensor responsive to the subject eating and an electrical current for tissue innervation following such sensing. However, such an approach which is limited to electrical stimulation of the vagal nerve endings has limited success.

Prior art studies revealed that CCK sensitizes gastric and duodenal mechanosensitive vagal afferents (van de Wall et al., 2005, Am. J. Physiol. Regul. Integr. Comp. Physiol. 289: R695-R703, and references therein) and that CCK infusion and gastric distension interact to reduce food intake (Kissileff H R., et al., 2003, Am. J. Physiol. Regul. Integr. Comp. Physiol. 285: R992R998).

Although prior art documents disclose several approaches for controlling food consumption or the urge to eat, none disclose an approach which combines monitoring of appetite or feeding with an efficient control of food consumption or absorption in a manner mimicking the physiologic pathway of appetite control and gastric emptying in the intestine.

While reducing the present invention to practice, the present inventor has devised an apparatus and a method that efficiently control food ingestion and/or appetite by mimicking the natural pathway of appetite control. The apparatus and/or method disclosed herein activate the chemoreceptors, the mechanoreceptors, and optionally also the electrical stimulation that regulate vagal innervation and induce satiety. The apparatus may utilize a drug releasing mechanism capable of releasing, in a timely fashion, a satiety drug such as CCK or analogs thereof at the site of its action, e.g., the duodenum, an inflatable balloon capable of space occupying and activation of the stomach mechanoreceptors and optionally electrodes capable of vagal innervation. Such an apparatus and/or method maximize the vagal-mediated satiety signals using specific stimulation by endocrine pathways (i.e., chemical stimulation), mechano-stimulated pathways and possibly specific electric pathways. In addition, as is further described hereinbelow, such a control over food ingestion and/or appetite is coordinated with sensing of food ingestion or hunger thereby achieving satiety shortly after sensing of hunger or food ingestion. Thus, when synchronized with food ingestion, the apparatus and the method of the present invention augment the physiologic pathways of satiety and achieve full satiety signaling at the beginning of a meal, leading to appetite curbing, early meal termination and reduction of food consumption.

Thus, according to one aspect of the present invention there is provided an apparatus for treating a weight disorder in a subject.

The apparatus includes an implantable device capable of sensing a physiological change associated with food ingestion or hunger and a mechanism adapted for directly stimulating a region responsive to a gastrointestinal satiety agent.

As used herein the phrase “weight disorder” refers to any pathology, disease, disorder or condition which leads to an abnormal BMI. The term “obesity” refers to a condition in which an individual's body mass and/or body fat is above the normal levels accepted for the individual's height, age and/or gender. Various parameters are used world-wide to determine the recommended body weight, the amount of body fat and the ranges beyond which a person is considered obese or over-weight. According to one parameter, a person is considered obese if he or she has a body mass index (BMI) of 30 kg/m² or greater. According to this parameter, mild obesity is defined by a BMI of 30-35 kg/m², severe obesity is defined by a BMI of 35-40 kg/m² and morbid obesity as BMI>40 kg/m². Other accepted parameters calculated the desired weight on an individual based on the individual's age, height and gender. It will be appreciated that weight disorder or obesity can lead to a metabolic syndrome such as a lipid disorder, visceral obesity, hypertension and dysglycemia (e.g., diabetes).

Obesity or over-weight can result from various pathologies, syndromes, diseases or disorders involving genetic factors (e.g., chromosomal abnormalities, mutations, or genetic predisposition) as well as environmental factors (e.g., social behavior, cultural customs, availability of rich and fat food and the like).

Although the present apparatus is best utilized with weight disorders such as obesity, it will be appreciated that disorders that lead to abnormally low BMI, such as anorexia can also be treated with the present apparatus.

The term “treating” as used herein, refers to inhibiting, preventing, curing, reversing, attenuating, alleviating, minimizing, suppressing or halting the deleterious effects of a condition and/or causing the reduction, remission, or regression of the condition (e.g., obesity or being over-weight). Those of skill in the art will understand that various methodologies and assays can be used to assess the development of a condition, and similarly, various methodologies and assays may be used to assess the reduction, remission or regression of a condition.

The terms “subject” or “individual” are interchangeably used herein to refer to a mammal, preferably a human being which suffers from a weight disorder or is predisposed thereto. Non-limiting examples of predisposed individuals include individuals who are genetically predisposed to develop a pathology which leads to a weight disorder (e.g., individuals who carry a mutation or a DNA polymorphism which is associated with high prevalence of the pathology), and/or individuals who are at high risk to develop the pathology or condition due to non-genetic factors (e.g., environmental factors as described hereinabove).

As used herein the phrase “implantable device” refers to a device being connected or contained within at least part of an individual's body. For example, such a device can be inserted into a cavity of the individual such as the gastric cavity (e.g., stomach, esophagus, intestine, duodenum) or the brain cavity (e.g., ventricle), or be connected with or implanted in any tissue of the individual (e.g., stomach, duodenum, stomach wall, skin, percutaneous, subcutaneous, esophagus and peritoneum).

As is mentioned hereinabove, the apparatus of the present invention includes an implantable device which is capable of sensing a physiological change associated with food ingestion or hunger.

A physiological change associated with food ingestion or hunger can be any physiological function of the body which is related to, caused by, or associated with food ingestion or hunger. Examples include muscle activity (e.g., of the stomach), pressure (e.g., resultant of entrance of food into the esophagus or the stomach), release of digestive juice including acid and enzymes (i.e., change in pH), body temperature, and electric current in the vagus or pancreas. Changes associated with hunger include release or suppression of hormonal signals to hunger like Ghrelin (GenBank Accession No. Q9UBU3).

Examples of devices which can be used with the present apparatus for sensing a physiological change associated with food ingestion or hunger can be an inflatable balloon capable of sensing the pressure formed by entrance of food into the esophagus or the stomach, an electrode capable of sensing muscle contraction (i.e., muscle activity) in the esophagus and/or the stomach, a pH-sensitive device (e.g., an electrode) which is capable of monitoring or sensing changes in pH in the stomach formed as a result of food consumption or hunger and/or a body temperature-sensitive device (e.g., as described in U.S. Pat. Appl. No. 20050096637A1 which is fully incorporated herein by reference).

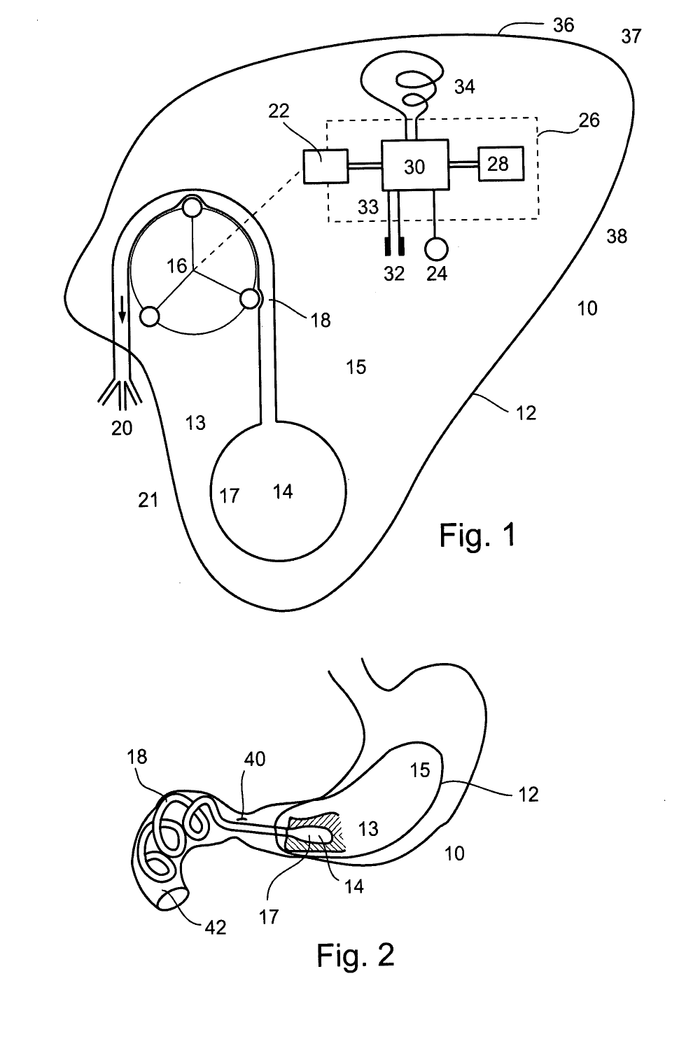

The inflatable balloon of the present invention which is capable of sensing the pressure formed by entrance of food into the esophagus or the stomach can be any inflatable gastric balloon known in the art which is used as a barrier in gastric surgeries (e.g., the BioEnterics® intragastric balloon). Such a balloon preferably includes a pressure sensor positioned within the balloon. A pressure sensor can include a microelectromechanical system with an array of sensors with pressure sensitivity, e.g., from 0.01 mmHg to a maximum pressure of 30 mmHg. Non-limiting examples of suitable pressure sensors include the pressure sensor lead of the Chronicle® (Medtronic) and the implantable pressure sensors available from Integrated Sensing Systems, Inc. Ypsilanti, Mich. It will be appreciated that such an inflatable balloon can be implanted into any gastric cavity such as the stomach, essentially as described in U.S. Pat. No. 4,485,805. Further description of the balloon which can be used along with the present invention is provided in the description of FIGS. 1-2, hereinbelow.

Additionally or alternatively, the implantable device of the present invention can be a pressure sensor which detects the pressure formed in the esophagus while food is ingested. Such a pressure sensor can be a microelectrode implanted under the esophagus endothelium that is connected, either directly or via radiofrequency transmission to a receiver (e.g., the electronic control unit shown in FIG. 1) which monitors food ingestion.

Electrodes capable of sensing muscle activities according to the present invention can be any electrode used in an EMG sensor such as those described in Lindsey D P., et al., 1998 (IEEE Trans Biomed Eng. 45: 614-9), US Pat. Appl. No. 20040220633 to Wagner, Darrell Orvin et al., and references therein, Pehlivan M., et al., 1996 (An electronic device measuring the frequency of spontaneous swallowing: Digital Phagometer; Dysphagia. 1996, 11: 259-64), all of which are fully incorporated herein by reference. EMG sensor are available from Delsys Incorporated and Nihon Kohden). Such electrodes or sensors can be implanted inside, outside or in near the stomach and/or the esophagus, such as in the coniotomy region between the cricoid and thyroid cartilage. It will be appreciated that such an electrode can be connected to the stomach or esophagus wall from the inner or outer side of the wall, or can be implanted percutaneously.

The implantable pH-sensitive device which is used along with the present invention can be, any pH-sensitive device known in the art which is suitable for implantation into the body. Non-limiting examples of such a device is the pH biotelemetry transmitter developed by NASA Ames Research Center and the Bravo™ pH Monitoring System (Medtronic). Such a pH-sensitive device can sense increase in gastric acidity due to a release of gastric juice. It will be appreciated that such a pH-sensitive device can be physically or remotely (via radio frequency) connected to a digital processor capable of analyzing the changes in pH values within the stomach as a function of food ingestion or hunger.

Further description of devices suitable for sensing a physiological change associated with food ingestion or hunger is provided hereinunder with respect to FIGS. 1-3.

As is mentioned hereinabove, the apparatus of the present invention also includes a mechanism adapted for directly stimulating a region responsive to a gastrointestinal satiety agent.

As used herein, the phrase “directly stimulating” refers to activating a response to a gastrointestinal satiety agent in a region, site or locus that is physiologically responsive to such agent.

As used herein, the phrase “a region responsive to a gastrointestinal satiety agent” refers to any region that includes a cell or a tissue being responsive to a gastrointestinal satiety agent (e.g., a cell having a receptor to a gastrointestinal satiety agent). It will be appreciated that such a region can be part of the gastrointestinal system and/or the central nervous system (CNS). Non-limiting examples of such a region include the duodenum, the antral sphincter, the intestine, small bowel, the hypothalamus and the liver. The phrase “gastrointestinal satiety agent” refers to any synthetic or naturally occurring hormone, peptide and/or neurotransmitter which is capable of inducing satiety. Such a satiety agent is produced by, secreted from and/or activates a response in cells of the gastrointestinal tract or the region in the CNS that controls satiety as part of the physiological response to food ingestion.

It will be appreciated that stimulation of the region responsive to the gastrointestinal satiety agent can be achieved using a drug that mimics the physiological action of the gastrointestinal satiety agent. Such a drug can be a naturally occurring or synthetic hormone, peptide, neurotransmitter or mimetic thereof, that is capable of directly stimulating the region responsive to the satiety agent (e.g., duodenum and small bowel). Non-limiting examples of such drugs include CCK (GenBank Accession No. NP_{—}000720; SEQ ID NO:1; van de Wall et al., 2005), CCK derivatives such as CCK-4 (Trp-Met-Asp-Phe; SEQ ID NO:2) and CCK-8 (Asp-Tyr(SO₃H)-Met-Gly-Trp-Met-Asp-Phe; SEQ ID NO:3), CCK analogues [Sincalide (Bracco Diagnostics, or Squibb Diagnostics), GSK-GW7176, GW 5283, GW7854 and Pfizer PW170292)], CCK receptor agonist (e.g., 1,5-benzodiazepines, PD 170292, SR 146131) and/or activator molecules of the CCK-A receptor (JMV 180; Archer-Lahlou E, et al., 2005, J. Biol. Chem., Vol. 280: 10664-10674), GLP-1 [Bojanowska E., 2005, Med. Sci. Monit. 11:RA271-8; BYETTA™ (exenatide)], PYY (le Roux C W., et al., 2005, Endocrinology. 2005 Sep. 15; Epub ahead of print; GenBank Accession No. NP_{—}004151; SEQ ID NO:4), PYY analog [e.g., PYY(1-36), PYY(3-36), PYY(9-36), PYY(14-36), PYY(22-36), and PYY(27-36)], Oxyntomodulin (OXY, OXM; GenBank Accession No. P01275; Stanley S., et al., 2004, Am. J. Physiol. Gastrointest. Liver Physiol. 286(5): G693), Apo IV (naturally occurring apoprotein Qin X, Tso P 2005, Curr Drug Targets. 6(2):145-51), GII81771X (GSK), anti Ghrelin agents (Kobelt P., Gut. 2005 Jun. 30; Epub ahead of print; SPIEGELMER NOX-B11), PP (Miskowiak J, et al., 1985, Regul. Pept. 12: 231-6). Of note is that the use of peptides like CCK might necessitate the addition of peptidase inhibitors such as thiorphan [((RS)-2-Benzyl-3-mercaptopropionyl)-Gly-OH ((DL-3-Mercapto-2-benzylpropanoyl)-Gly-OH; DL-Thiorphan Bachem)] and amastatin ([(2S,3R)-3-Amino-2-hydroxy-5-methylhexanoyl]-Val-Val-Asp-OH HCl; Bachem), which are specific inhibitors of enkephalinase and aminopeptidase, respectively, in order to maximize action and absorption potential. Larger peptides may also require use of absorption enhancers and surfactants [for example Labrasol Labrasol® (PEG-8 caprylic/capric glycerides, HLB value of 14 Gattefosse (Lyon, France)]. It will be appreciated that other drugs which are capable of preventing food absorption such as lipase inhibitors (e.g., orlistat; Xenical), can be also used along with the present invention. Methods of synthesizing the peptides and analogues thereof of the present invention are further described hereinunder.

According to this aspect of the present invention the mechanism adapted for directly stimulating the region responsive to the gastrointestinal satiety agent is designed to release the drug in close vicinity to the cell or tissue (e.g., in a cavity containing such cells) which is responsive to the gastrointestinal satiety agent (i.e., the drug's target cell or tissue). For example, such a satiety drug can be released into the duodenum, antral sphincter, intestine or any natural locus of action in the gastrointestinal system and in the central nervous system (e.g., the hypothalamus). Specifically, such a satiety drug can be released close to the duodenum wall (i.e., near the duodenum mucosa, e.g., within 0.1-3 mm of the duodenum mucosa) or another wall of the GI system encompassing chemoreceptors and afferent nerve endings. Delivery of such a drug into the duodenum is likely to provide an enhanced or synergistic therapeutic effect due to its local effect on the mucosa. Specifically, CCK analogs can be delivered to interact with CCK-α receptors in the peripheral vagal endings in the area of the duodenum and pyloric sphincter. Additionally or alternatively such agents can be absorbed and taken up into the local blood stream, delivered through the portal circulation into their peripheral or central locus of action. Gastrointestinal satiety factors like GLP-1, PYY or anti-Ghrelin agents (which suppress the feeling of hunger) can be administered to their action sites in and out of the gastrointestinal (GI) tract in a timely physiologic fashion.

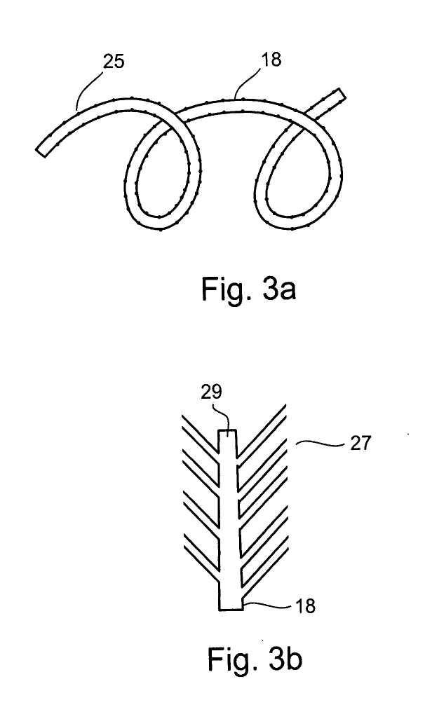

Preferably, the mechanism adapted for directly stimulating a region responsive to a gastrointestinal satiety agent is a drug-releasing mechanism. Such a drug-releasing mechanism can be, for example, a drug reservoir. The drug-releasing mechanism of the present invention can be implanted in the body of the subject to be treated (e.g. in the stomach or the small intestines), percutaneously, or outside the body. It will be appreciated that a drug-releasing mechanism which is implanted in the body can be placed inside or near an inflatable object such as an inflatable balloon. In case the drug-releasing mechanism is implanted percutaneously or placed outside the body a fluid conduit (i.e., a dispersing tube) such as a catheter or a cannula can connect the mechanism with the site of drug delivery. The design of the dispersing tube is such that ensures the proximity of drug delivery to the mucosa of the GI wall (e.g., to a distance of 0.1-3 mm from the mucosa of the duodenal or other GI wall). Further description of such a dispersing tube is provided hereinbelow with respect to FIGS. 3a-b.

The drug reservoir used by the present invention can be a replaceable reservoir or a reloadable (e.g., replenished) reservoir. Non-limiting examples drug reservoirs which can be used along with the apparatus of the present invention are described in U.S. Pat. Nos. 6,048,328 and 20020087113, 20010041870, all of which are fully incorporated herein by reference. For example, replenishing of the drug can be effected by inserting a non-coring needle connected to a syringe filled with the drug, essentially as described in U.S. Pat. Appl. No. 20020087113.

Various approaches can be used to introduce the drug reservoir of the present invention into the body, including endoscopy and percutaneous gastrostomy (see for example, U.S. Pat. Appl. No. 20050143691 and 20010037127 and references therein). The reservoir can be introduced in a collapsed manner and introduced endoscopically to the stomach. After localization it can be filled with the appropriate drug.

According to one preferred embodiment of this aspect of the present invention, the reservoir is positioned inside an inflatable balloon which is implanted in the body as described hereinabove. When this configuration is utilized, the release of the drug from the reservoir into the gastric cavity (e.g., duodenum or small bowel) is effected using a cannula or a catheter which penetrates through the balloon and thus connects the drug reservoir with the gastric cavity.

The reservoir may be connected to a pump, or can be in a form of a pump. Such a pump can be osmotic, electrically (using internal or external energy sources) or mechanically driven, possibly using a semipermeable coating or a hygroscopic material. The pump can be positioned in a sealed housing in order to keep a dry environment for the electronic components included therein. The pump could provide means to control the delivery protocol (timing, rates, doses, profiles etc.) of the drug of the present invention, using for example controllers, valves or additional systems known in the arts.

The control of the pump may be internally by a programmable setup (as is customary with insulin pumps), or externally through wireless communications (as is found in some pacemakers and defibrillator devices, e.g., by Medtronic). It could be activated manually or automatically as the hunger drive or food consumption occurs. Such application will curb appetite and limit meal size.

Non-limiting examples of suitable pumps which can be used along with the present invention include, an osmotic pump (e.g., as described in Yu-Chuan Su et al., J. Microelectromechanical Systems, 2004, 13:75, A Water-Powered Micro Drug Delivery System), an electrical pump such as a battery-powered, programmable drug delivery system (see for example, Vogelzang N. J., et al., 1985, Journal of Clinical Oncology, 3: 407-414), the Medtronic SynchroMed Infusion System, the Medtronic MiniMed pump (MiniMed Paradigm Family), Disetronic D/Htrons Family (Roche), Animas or Deltec insulin pumps or the, a mechanical pump and a peristaltic pump (Medtronic).

The pump can deliver the drug to the site of action along the GI tract through a tube or an opening (depending on the location). The tube preferably ends with a delivery device that ensures even distribution of the drug to the appropriate target site. Such a delivery device may include hooks (e.g., the ones used for venous filters), microneedles (e.g., Spectrx insulin microneedles), microprojections, and canulas (e.g., available from Becton Dickinson and Co.). The pump can enable various delivery protocols such as continuous or intermittent. Further description of the pump and the drug delivery means is provided in FIGS. 1 and 3.

It will be appreciated that the apparatus of the present invention can further include a distending mechanism capable of stimulating vagal mechanoreceptors such as a distending object (e.g., an inflatable object, mesh, spring or coil) or a space-filling structure (e.g., an inflatable balloon which occupies part of the stomach space and thus limits the space which can include food). Thus, in addition to activation of the vagal chemoreceptors by local delivery of the satiety drug (e.g., CCK or analogs thereof), the apparatus of the present invention exerts a synergistic effect on satiety which can efficiently curb appetite and reduce meal size. An inflatable object (e.g., an inflatable balloon) can be filled with air, saline, water or other means and can be positioned within the stomach of the subject. While expanded, and especially following entrance of food into the stomach, such an inflatable object (e.g., a balloon) exerts a significant pressure on the mechanoreceptors present on the inner side of the stomach wall, which further activate vagal innervation and induce satiety [van de Wall, 2005 (Supra). See also: Innomed BIB Balloon, http://www.inamed.com/pdf/health/BIB_Bibliography_{—}10_{—}19_{—}00.pdf]. Space-filling structures can be made of a biodegradable polymer and/or form a hydrogel. Such space-filling structures can be safely inserted into the stomach and lodged therein. It will be appreciated that the distending mechanism can keep the drug reservoir in the stomach and prevent slippage thereof through the antral sphincter.

Additionally or alternatively, the apparatus of the present invention can further utilize an electrode capable of vagal innervation along with the mechanism capable of directly stimulating the region responsive to a gastrointestinal satiety agent. It will be appreciated that by stimulating vagal chemoreceptors (using the local delivery of the satiety drug) and by directly innervating the specific vagal nerve pathways, a combined satiety effect, which stimulates an afferent arm, is achieved. Preferably, a mechanism for electrical vagal innervation utilizes at least one electrode. More preferably, such a mechanism includes at least 2 electrodes, more preferably, at least 3, more preferably, at least 4, more preferably, at least 5 electrodes. Suitable electrodes which can be used along with the present invention are described in U.S. Pat. Appl. No. 20050192644 to Boveja, Birinder R., et al., and U.S. Pat. Appl. No. 20040059393 which are fully incorporated herein by references.

Similarly, the electrodes used by the apparatus of the present invention can be used to conduct electrical signals to Gherlin-secreting cells in the stomach. Such electrical signals can be given at specific locations in the GI tract and using specific frequencies and strength selected capable of preventing Gherlin secretion and thus overcoming the feeling of hunger. It will be appreciated that such electrical stimulation is preferably effected in a coordinated manner with sensing of hunger.

As is mentioned hereinabove, electrode(s) used according to this aspect of the present invention (for either sensing or stimulating) can be positioned within the gastric cavity (e.g., inside the stomach, by disposing on any implantable device) or percutaneously (i.e., outside of the gastric cavity) and positioned adjacent or in the gastric muscle. Such electrodes can be connected to an electrical source, preferably with a control unit capable of controlling vagal innervation.

According to one preferred embodiment of this aspect of the present invention, the electrode capable of vagal innervation is disposed on an inflatable object (e.g., a balloon) implantable within a stomach of the subject.

It will be appreciated that the mechanism adapted for directly stimulating a region responsive to a gastrointestinal satiety agent can be constantly active, active according to a pre-determined schedule, active according to an outer signal (e.g., provided by the subject being treated) or can be coordinated with the device which senses food ingestion or hunger such that stimulation of the region responsive to the GI satiety agent takes place shortly after sensing food ingestion or hunger, thus reducing the amount of food intake.

For example, a muscle activity sensor which is capable of sensing the peristaltic movement of the esophagus or the stomach (following food ingestion) can be connected to a digital processor capable of calculating the volume and/or consistency of the food ingested by recording the number of peristaltic movements of the esophagus or the stomach. Following such calculation, the processor can control drug release and optionally induce electrical vagal innervation to achieve a level capable of suppressing appetite, reducing the drive or desire to continue eating, and limiting meal size.

Alternatively or additionally, a pressure sensor which is placed inside an inflatable balloon can monitor the change in pressure which follows food ingestion into the stomach or esophagus and can transduce such a change into a signal received by a digital processor, which is capable of controlling the release of the drug and optionally inducing electrical vagal innervation.

Still additionally or alternatively, an electrode which is capable of sensing muscle activity and is disposed on the inflatable balloon as described hereinabove, can be connected to a digital processor which can control the release of a drug from a drug reservoir and optionally inducing electrical vagal innervation using the same electrode.

FIGS. 1-3 describe in detail several preferred embodiments of the apparatus of the present invention which is referred to herein as apparatus 10.

FIG. 1 illustrates apparatus 10 which includes device 15 that is capable of sensing physiological changes associated with food ingestion and/or hunger and mechanism 13 adapted for directly stimulating a region responsive to a gastrointestinal satiety agent. Device 15 includes balloon 12, a pressure sensor 24 and at least one electrode 32 for detecting stomach muscle activity indicative of hunger, feeding or satiation.

Balloon 12 is positionable within a stomach of a subject (e.g. human) and is selected of a size such that when collapsed it can be orally introduced into the stomach (via, for example, endoscopy) and when expanded it can sense stomach contraction, stomach muscle function and/or stomach filling (e.g., with food) and yet limit stomach capacity without interfering with stomach function (e.g. digestion).

According to one preferred embodiment of the present invention, balloon 12 is selected of a shape and size such that when expanded (e.g., inflated), and especially following food ingestion, balloon 12 is capable of activating the mechanoreceptors in the stomach wall and thus activate vagal innervation and satiety, which curb appetite. Balloon 12 can be made of any material that will not deteriorate in the stomach or interfere with its activity. Balloon 12 is preferably made of a distensible material such as a medical grade silicone elastomer (e.g., Silastic), polyurethane, latex rubber and the like. Preferably, balloon 12 is formed of a puncturable, yet resealable material (i.e., a material which can reseal following puncturing).

In its collapsed state, balloon 12 may have the following dimension: length-10-20 cm, diameter 1-3 cm. Balloon 12 can be deployed by endoscopy using methods currently practiced in clinics for endoscopy. Balloon 12 can be expanded with any fluid including saline or air, using a filling tube which is disconnected from balloon 12 following expansion. Balloon 12 may have an opening 36 which communicates with a filling tube 37 which can be fastened by suitable adhesives to form an airtight seal. Alternatively, balloon 12 can be self inflatable via a gas forming substance (e.g., potassium or sodium bicarbonate) that reacts with the stomach acid (e.g., citric acid) or by covering the balloon with a haemostatic agent, such as a knitted fabric manufactured from carboxymethylatedcellulose (CMC) which forms a gel after contacting with blood or other fluids such as mucus (see International Patent Application No. PCT/GB00/03586 which is incorporated herein by reference).

Once expanded, the dimension of balloon 12 should be chosen so that a distension to a diameter of at least 20 centimeters (cm) would result if the balloon was filled with fluid to an internal pressure of about 20 millimeters of mercury (mmHg) outside the subject in ambient air. Preferably, when inflated, balloon 12 has a capacity between about 200 to about 1000 ml and may have a ball-shape or a cylinder shape. It will be appreciated that these dimensions are suitable for an adult human subject and may be scaled proportionally for younger subjects or different mammals.

Pressure sensor 24 is positionable inside balloon 12 and is designed for detecting changes in pressure following entrance of food into the stomach. Pressure sensor 24 is made of an array of sensors with increasing pressure sensitivities and can detect the pressure inside balloon 12. Once food is ingested into the stomach, balloon 12 is compressed and the pressure inside increases. Such changes in the pressure inside balloon 12 can be detected by sensor 24 and are further transduced to electrical signals. Such electrical signals are transferred to electronic control unit (ECU) 30 which is preferably contained within balloon 12. ECU 30 serves to process signals received from sensor 24 and electrodes 32.

Electrodes 32 can be EMG sensor electrodes are made of a material resistant to stomach acid such as platinum, titanium and/or stainless steel. Electrodes 32 are positioned such that they electrically contact surrounding tissue to thereby detect muscle activity in the stomach and/or the esophagus. Electrodes 32 can detect the strength of muscle contraction (measured in mV units) as well as the number of contraction over a time period to determine contraction rate (e.g. contractions per minutes). Electrodes 32 transfer electrical signal to ECU 30 via electrical leads 33.

Mechanism 13 adapted for directly stimulating a region responsive to a gastrointestinal satiety agent can be attached to balloon 12 or incorporated thereinto.

Mechanism 13 includes a drug reservoir 14, a pump 16 (e.g. peristaltic), a delivery cannula 18 and a diffuser 20 at the cannula distal end.

Drug reservoir 14 has an internal volume in the range of 5-1000 ml. For example, such a volume can be between about 5-600 ml, 100-600 ml, 200-500 or even between about 5-200 ml. Drug reservoir 14 is fabricated from a suitable material for containing drug 17. Drug 17 may include one type of drug or any combination of drugs suitable for inducing satiety. Preferably, drug 17 is CCK, CCK-4, CCK-8, CCK analogues or a CCK receptor agonist. Drug reservoir 14 is designed such that it can release drug 17 at the region responsive to a gastrointestinal satiety agent. Preferably, drug reservoir 14 is selected capable of releasing drug 17 (e.g., CCK or analogs thereof) at the duodenum, to thereby activate the duodenal chemoreceptors, induce vagal innervation and satiety. Drug reservoir 14 is formed of a re-sealable material which will reseal when a needle used for replenishing drug reservoir 14 is withdrawn. A delivery cannula 18 is connected to drug reservoir 14 for delivering drug 17, through a diffuser 20 at the cannula distal end. Deliver cannula 18 is made of silicon, polyurethane or any biocompatible polymer such as Teflon (see for example, BD Venflon™) and has a length of about 5-30 cm and a diameter of about 0.5-3 mm. Diffuser 20 is made of silicon, polyurethane or any biocompatible polymer and can include multiple outlets (for example 2-10), each of a length of 2-20 mm and a diameter of 0.2-1 mm.

Pump 16 allows the release of drug 17 from drug reservoir 14. Pump 16 is designed capable of releasing a drug according to a command provided from ECU 30. Pump 16 is housed in a sealed housing 26 which also houses a power source 28 (e.g. a battery) and a motor unit 22.

Sealed housing 26 can be made of polymer such as titanium and can be of various sizes. Sealed housing is selected of a volume necessary to house pump 16, power source 28 and motor unit 22 described above, as well as ECU 30. For example, sealed housing 26 can be of the following dimensions: diameter 2-12 cm and width 1-3 cm.

Motor unit 22 is capable of driving peristaltic pump 16 to enable delivery of drug 17 from drug reservoir 14, through cannula 18 to diffuser 20 and the target tissue 21. Motor unit 22 receives electrical signal from ECU 30 which controls its activity.

ECU 30 receives electrical signals from device 15 which includes the input obtained from sensor 24 (which senses the changes in pressure inside balloon 12) and/or the input obtained form electrodes 32 (which sense frequency and strength of muscle activity in the esophagus and/or the stomach). ECU 30 can be a digital processor which analyses the input (i.e., electrical signals) and calculates, according to a software program stored and executed thereby, the desired dose of drug 17 to be released from drug reservoir 14 and/or the level of vagal innervation to be produced from electrodes 32. ECU 30 sends its output (i.e., electrical signal) to electrodes 32 and motor unit 22.

Mechanism 13 can also include a coil 34 to re-charge power source 28 and program the pump parameters using a remote controller (i.e., a transmitter receiver). Coil 34 can be a magnetic or a radio frequency activated coil capable of re-charging power source 28 and is made of a conducting material such a copper, gold.

FIG. 2 illustrates positioning of apparatus 10. In this example, device 15 for sensing food ingestion or hunger includes balloon 12 (which can include a pressure sensor as described hereinabove) positioned in the stomach near the Pylorus 40. Apparatus 10 also includes mechanism 13 adapted for directly stimulating a region responsive to a gastrointestinal satiety agent. Mechanism 13 includes drug reservoir 14 which is positioned inside balloon 12 and is connected to a cannula 18 which extends from mechanism 13 to the surface of balloon 12 and is capable of releasing drug 17 into the duodenum 42.

FIGS. 3a-b illustrate two exemplary cannula configurations which can be used with apparatus 10 of the present invention. FIG. 3a depicts a dispersing tube which comprises multiple delivery holes 25 through which drug 17 can be dispersed in a preferred location. Holes 25 have a diameter in the range of 0.1-1 mm. FIG. 3b depicts a cannula 18 configuration which includes several delivery tubes 27 branching from a main tube 29 which is connected to drug reservoir 14. The diameter of main tube 29 is about 0.5-3 mm and the diameter of each of delivery tubes 27 is about 0.1-1 mm. It will be appreciated that cannula 18 can include 2-25 delivery tubes 27.

Although an apparatus which is capable of sensing food ingestion or hunger is presently preferred, the present invention also envisages the use of a device capable of stimulating a region responsive to a gastrointestinal satiety agent in a constant or a pre-programmed manner without need for pre-sensing stomach activity.

Such a device can include a distending object such as an inflatable balloon and a drug reservoir for releasing the drug of the present invention (e.g., CCK or analog thereof). The drug reservoir can be positioned in or outside the distending object (e.g., the balloon) and release the drug in a local manner to the duodenum, antral sphincter, or in a close proximity (i.e., 0.1-3 mm) to a mucosa of any GI wall. Programming of drug release can be made using a controller which sends signals using radio frequency. It will be appreciated that such apparatus may be used to treat obese individuals which require constant levels of satiety drugs in order to limit food consumption (e.g., morbid obese individuals).

The apparatus of the present invention can be used to treat weight disorder in a subject. Thus, according to another aspect of the present invention, there is provided a method of treating a weight disorder. The method is effected implanting in a subject in need thereof a device capable of sensing a physiological change associated with food ingestion and/or hunger; and functionally associating with the device, a mechanism adapted for directly stimulating a region responsive to a gastrointestinal satiety agent for modifying appetite of the subject.

As used herein the phrase “functionally associating” refers to combining or connecting, either physically or by remote control, the function of the mechanism adapted for directly stimulating a region responsive to a gastrointestinal satiety agent with the device capable of sensing food ingestion and/or hunger.

As is mentioned hereinabove, the mechanism adapted for directly stimulating a region responsive to a gastrointestinal satiety agent can be either implanted in the body or be placed outside of the body. According to one preferred embodiment of this aspect of the present invention, such a mechanism is implanted along with the device capable of sensing food ingestion or hunger.

Preferably, stimulation of the region responsive to GI satiety agent by the release of the drug and optionally also and in parallel by inducing electrical vagal innervation is coordinated with sensing of food ingestion or hunger.

For example, such stimulation (e.g., release of drug at the duodenum) can be effected following a pre-determined time period after sensing of food ingestion or hunger. Such a time period can be at least 30 seconds, more preferably, at least 1 minute, more preferably, between 1-10 minutes, even more preferably, between 1-5 minutes after sensing of food ingestion or hunger.

The release of the satiety drug at the responsive region can be effected for various time periods effective to induce satiety and limit meal size, and those of skills in the art are capable of calculating such time periods. For example, the drug can be released at the start of a meal (e.g., following 2 minutes of sensing food ingestion) by a bolus administration of the drug delivered for 1-5 minutes. Following the initial bolus, a lower dose of drug can be delivered at a constant flow of up to 20 minutes. Such administration method can prevent resurge of hunger.

Similarly, the electrical stimulation of the vagal nerve endings (i.e., vagal innervation) using the electrodes described hereinabove can be effected in a constant [i.e., all the time using constant (e.g., low) frequency], pre-programmed schedule (e.g., for 1-3 hours, three times a day), timely-coordinated with food ingestion. For example, vagal innervation can begin after sensing food ingestion or hunger, e.g., following 1-5 minutes of such sensing and can last for e.g., 30-90 minutes.

Thus, the apparatus and/or the method of the present invention provide, for the first time, an efficient approach for treating obesity. The combination of a device capable of sensing food ingestion or hunger with a mechanism adapted for directly stimulating a region responsive to a gastrointestinal satiety agent enables an efficient therapeutic approach for treating obesity and other weight disorders. The controlled release of the drug in a timely-coordinated fashion (e.g., following sensing of food ingestion or hunger) in its natural (or physiological) target site (i.e., where it is normally active to induce satiety) such as the duodenum, enables an efficient curbing of appetite and a significant limiting of food intake. In addition, since the drug is locally delivered, it will be appreciated that the dosage used to induce satiation is far lower than that used for systemic administration, thus preventing the drug's possible side effects. Thus, the apparatus of the present invention which combines a mechanical pressure (e.g., by a distended balloon), a limitation of stomach free space which is a principle of bariatric surgery and a local release of the drug is capable of delaying gastric emptying and appetite curbing, and thus preventing and treating obesity. It will be appreciated that the combination of electrical stimulation of vagal innervation with releasing of the drug at the site of action (like antral sphincter or duodenum) result in activation of vagal innervation both locally on mucosa and vagal nerve endings and remotely through systemic absorption on the stomach and the brain. Altogether, the apparatus and/or method of the present invention combines chemo and mechano receptor activation of vagal satiety stimuli, electric stimulation of specific vagal pathways and limitation of gastric space and thus achieves a synergistic effect which limits meal size.

As is mentioned before, the drug of the present invention can be a peptide or mimetic thereof which acts as a satiety or anti-food absorption drug.

The term “peptide” as used herein encompasses native peptides and their analogues (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The peptides of the present invention are preferably utilized in a linear form, although it will be appreciated that in cases where cyclicization does not severely interfere with peptide characteristics, cyclic forms of the peptide can also be utilized.

The peptides of the present invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of the present invention involves solid phase peptide synthesis.

Large scale peptide synthesis is described by Andersson Biopolymers 2000; 55(3):227-50.

The drug used by the present invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the satiety drug accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include into a gastric cavity (e.g., stomach, duodenum, intestine), into a gastric tissue, and into the CNS (e.g., into the ventricular cavity).

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

Alternatively, the active ingredient may be in powder form for constitution with a suitable vehicle, e.g., sterile, pyrogen-free water based solution, before use.

Pharmaceutical compositions suitable for use in context of the present invention include compositions wherein the active ingredients are contained in an amount effective to achieve the intended purpose. More specifically, a therapeutically effective amount means an amount of active ingredients (e.g., a satiety drug or an anti-food absorption drug) effective to prevent appetite.

Determination of a therapeutically effective amount is well within the capability of those skilled in the art, especially in light of the detailed disclosure provided herein.

For any preparation used in the methods of the invention, the therapeutically effective amount or dose can be estimated initially from animal models such as monkey or pigs. For example, a dose can be formulated in animal models to achieve a desired concentration or titer. Such information can be used to more accurately determine useful doses in humans.

For example, a bolus injection of CCK-8 can be in the range of 0.04-0.4 μg per kg body weight. Thus, for an individual who weighs 125 kg, such a bolus injection can be for example of 10 μg CCK-8. It will be appreciated that an efficient dose can be adjusted to the treated individual based on clinical trials and the degree of obesity.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide local and central levels of the active ingredient which are sufficient to curb appetite (minimal effective concentration, MEC). The MEC will vary for each preparation, but can be estimated from animal models. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Additional References are Cited in Text

U.S. Pat. No. 6,627,206 to Lloyd Sep. 30, 2003, Method and apparatus for treating obesity and for delivering time-released medicaments.
U.S. Pat. No. 4,694,827 to Weiner, et al. Filed 1986, Inflatable gastric device for treating obesity and method of using the same.
U.S. Patent Application, Publication No. 20030167024 to Imran, Mir A. et al. Sep. 4, 2003, Gastric device and instrument system and method.
U.S. Patent Application, Publication No. 20040148034 to Kagan, Jonathan; et al. Jul. 29, 2004, Apparatus and methods for treatment of morbid obesity.
U.S. Pat. No. 5,234,454 to Bangs Aug. 10, 1993, Percutaneous intragastric balloon catheter and method for controlling body weight therewith.
U.S. Pat. No. 5,795,887, Method of inducing cholecystokinin agonist activity using 1,4-Benzodiazepine compounds.
U.S. Pat. No. 6,579,852, OBG3 globular head and uses thereof for decreasing body mass.
U.S. Pat. No. 5,739,129 CCK or gastrin modulating 5-heterocyclic-1,5 benzodiazepines.
U.S. Pat. No. 6,675,809 to Stack, et al. Jan. 13, 2004, Satiation devices and methods.

These patent documents are hereby incorporated by reference as if fully set forth herein.

1-54. (canceled)

55. An apparatus for treating a weight disorder in a subject comprising an implantable device capable of sensing a physiological change associated with food ingestion or hunger and a mechanism adapted for directly stimulating a region responsive to a gastrointestinal satiety agent.

56. The apparatus of claim 55, wherein said device capable of sensing a physiological change associated with food ingestion or hunger comprises an inflatable balloon.

57. The apparatus of claim 55, wherein said device capable of sensing a physiological change associated with food ingestion or hunger comprises at least one electrode.

58. The apparatus of claim 55, wherein said implantable device capable of sensing said physiological change associated with food ingestion or hunger comprises a muscle activity sensor.

59. The apparatus of claim 55, wherein said mechanism comprises a drug reservoir being capable of containing and releasing a drug in response to said physiological change associated with food ingestion or hunger sensed by said implantable device.

60. The apparatus of claim 59, said mechanism configured to release said drug to a duodenum wall, to an antral sphincter and/or to a gastrointestinal wall.

61. The apparatus of claim 59, said drug reservoir positioned inside an inflatable balloon.

62. The apparatus of claim 59, further comprising an inflatable balloon implantable within a stomach of the subject, said inflatable balloon capable of activating vagal mechanoreceptors and/or space-filling.

63. The apparatus of claim 59, said mechanism further comprising at least one electrode capable of vagal innervation.

64. The apparatus of claim 63, wherein said at least one electrode capable of vagal innervation is disposed on a balloon implantable within a stomach.

65. The apparatus of claim 63, said mechanism configured to effect said vagal innervation 1-5 minutes following said sensing by said device capable of sensing a physiological change associated with food ingestion or hunger.

66. The apparatus of claim 55, configured for implantation in a stomach.

67. The apparatus of claim 55, said mechanism comprising an injectable device capable of injecting a drug.

68. A method of treating a weight disorder comprising:
a) implanting in a subject in need thereof a device capable of sensing a physiological change associated with food ingestion or hunger;
b) functionally associating with said device, a mechanism adapted for directly stimulating a region responsive to a gastrointestinal satiety agent; and
c) subsequent to sensing a physiological change associated with food ingestion or hunger by said device, activating said mechanism so as to directly stimulate said region.

69. The method of claim 68, wherein said stimulation of said region is effected using a drug.

70. The method of claim 69, wherein said drug is released to a duodenum wall, to an antral sphincter and/or to a gastrointestinal wall.

71. The method of claim 69, wherein said drug is a satiety drug and/or an anti food absorption drug.

72. The method of claim 68, wherein said mechanism comprises a drug reservoir containing a drug; and further comprising:
releasing said drug from said drug reservoir as said direct stimulation in response to said sensing by said device of said physiological change associated with food ingestion or hunger.

73. The method of claim 68, further comprising implanting in said subject an inflatable balloon capable of activating vagal mechanoreceptors and/or space-filling.

74. The method of claim 73, further comprising:
subsequent to sensing a physiological change associated with food ingestion or hunger by said device, activating vagal mechanoreceptors with said inflatable balloon.

75. The method of claim 68, wherein said mechanism comprises at least one electrode capable of vagal innervation; and further comprising:
performing vagal innervation with the help of said electrode.

76. The method of claim 68, wherein said device capable of sensing a physiological change associated with food ingestion or hunger comprises at least one electrode.

77. The method of claim 76, wherein said physiological change sensed comprises an electrical activity of a muscle.

78. The method of claim 68, wherein said implantable device capable of sensing said physiological change associated with food ingestion or hunger comprises a muscle activity sensor.

79. The method of claim 68, wherein said device capable of sensing a physiological change associated with food ingestion or hunger is implanted in a stomach of said subject.

80. The method of claim 68, wherein said need of said subject is related to a weight disorder selected from the group consisting of obesity, bulimia, diabetes-related obesity and a metabolic syndrome.

81. An apparatus for treating a weight disorder in a subject comprising an implantable device capable of sensing a physiological change associated with food ingestion or hunger and a mechanism adapted for directly stimulating a region responsive to a gastrointestinal satiety agent, said mechanism comprising an inflatable balloon implantable in a stomach of a subject and a drug reservoir containing a drug capable of stimulating said region, said drug selected from the group consisting of CCK, a CCK analog, a CCK receptor agonist, a PYY, a PYY analog, GLP-1, a GLP-1 analog and oxyntomudulin.

82. The apparatus of claim 81, said drug reservoir being capable of containing and releasing said drug in response to said physiological change associated with food ingestion or hunger.

83. The apparatus of claim 82, said mechanism configured to release said drug from said drug reservoir to a duodenum, to an antral sphincter and/or to an intestine.

84. The apparatus of claim 81, said device capable of sensing a physiological change associated with food ingestion or hunger comprises at least one electrode capable of sensing an electrical activity of a muscle.

85. The apparatus of claim 81, wherein said device capable of sensing a physiological change associated with food ingestion or hunger comprises a pressure sensor.

86. A method of treating a weight disorder comprising:
a) implanting in a subject in need thereof a device capable of sensing a physiological change associated with food ingestion or hunger;
b) functionally associating with said device a mechanism adapted for directly stimulating a region responsive to a gastrointestinal satiety agent, said mechanism comprises an inflatable balloon being implantable in a stomach of the subject and a drug reservoir containing a drug capable of stimulation of said region, said drug is selected from the group consisting of CCK, a CCK analog, a CCK receptor agonist, a PYY, a PYY analog, GLP-1 and GLP-1 analog; and
c) subsequent to sensing a physiological change associated with food ingestion or hunger by said device, releasing said drug from said reservoir.

87. The method of claim 86, wherein said physiological change associated with food ingestion or hunger comprises an electrical activity of a muscle.

88. The method of claim 86, wherein said physiological change associated with food ingestion or hunger comprises a change in pressure in a stomach of said subject.

89. The method of claim 86, wherein said drug is released to a duodenum, to an antral sphincter and/or to an intestine.

90. An apparatus for treating a weight disorder in a subject comprising a distending object being implantable in a stomach of the subject and a drug reservoir containing a drug capable of stimulation a region responsive to a gastrointestinal satiety agent.

91. A method of treatment, comprising:
a) providing a pharmaceutical composition which comprises a peptide drug;
b) releasing said drug to an antral sphincter and/or a gastrointestinal wall so that said drug activates chemoreceptors on said antral sphincter and/or a gastrointestinal wall
thereby causing a beneficial effect.

92. The method of claim 91, wherein said gastrointestinal wall is a duodenum wall.

93. The method of claim 91, wherein said releasing of said drug follows the sensing of food ingestion or hunger.

94. An apparatus for administering a drug to a luminal wall of a portion of the gastrointestinal tract, comprising:
a) a drug dispersing tube and configured to release a drug to an antral sphincter and/or a gastrointestinal wall;
b) a drug releasing mechanism functionally associated with a drug reservoir and with said drug dispering tube, configured to release a dose of drug held in said drug reservoir out through said dispersing tube; and
c) a pharmaceutical composition including a peptide drug held in said drug reservoir.

95. The apparatus of claim 94, said drug is configured to activate chemoreceptors on an antral sphincter and/or a gastrointestinal wall.

96. The apparatus of claim 94, wherein said drug is a synthetic or naturally occurring hormone, peptide, neurotransmitter or mimetic thereof.

97. A method of administering a drug, comprising:
a) deploying a drug dispersing tube of an apparatus of claim 94 in a location of a gastrointestinal tract of a subject suffering from a condition;
b) providing a pharmaceutical composition comprising a peptide drug, said drug effective in treating said condition; and
c) releasing said drug following sensing a physiological change through said dispenser so that said drug directly activates chemoreceptors on an antral sphincter and/or gastrointestinal wall
thereby administering said drug so as to treat said condition.

98. The method of claim 97, wherein said gastrointestinal wall is a duodenum wall.

99. The method of claim 97, wherein said drug is a synthetic or naturally occurring hormone, peptide, neurotransmitter or mimetic thereof.

100. The method of claim 97, wherein said drug is a satiety drug and/or an anti food absorption drug.
